(19) United States
(12) Patent Application Publication
(10) Pub. No.: US 2015/0119386 A1
(43) Pub. Date: Apr. 30, 2015

(54) PROCESS FOR PREPARING OPTHALMIC SUSPENSION OF BRINZOLAMIDE

(71) Applicant: AUROBINDO PHARMA LIMITED, Hyderabad (IN)

(72) Inventors: Chandrasekhar Kadam, Hyderabad (IN); Jayant Karajgi, Hyderabad (IN); Sachin Jain, Hyderabad (IN); Ajay Mhaske, Hyderabad (IN); Sivakumaran Meenakshisunderam, Hyderabad (IN)

(21) Appl. No.: 13/261,992

(22) PCT Filed: May 21, 2013
(86) PCT No.: PCT/IB2013/000986

§ 371 Date: Nov. 20, 2014

(30) Foreign Application Priority Data
May 21, 2012 (IN) 2003/CHE/2012

(51) Int. Cl. A61K 31/542; A61K 47/18; A61K 47/26; A61K 47/02; A61K 9/00; A61K 47/34

(52) U.S. Cl. CPC A61K 31/542; A61K 9/0048; A61K 47/34; A61K 47/26; A61K 47/02; A61K 47/183; A61K 47/186
USPC 514/226.5

(57) ABSTRACT

The present invention relates to a process for preparing sterile ophthalmic suspension of carbonic anhydrase inhibitor. More particularly, the present invention relates to a process for preparing sterile ophthalmic suspension of brinzolamide.

### FIELD OF THE INVENTION

The present invention relates to a process for preparing sterile ophthalmic suspension of carbonic anhydrase inhibitor. More particularly, the present invention relates to a process for preparing sterile ophthalmic suspension of brinzolamide.

### BACKGROUND OF THE INVENTION

Ophthalmic compositions of carbonic anhydrase inhibitors (CAI) are used to reduce elevated intraocular pressure in patients with ocular hypertension or open-angle glaucoma.

Oral CAIs such as oral acetazolamide have been a classical treatment for glaucoma but are very poorly tolerated because of systemic side effects. The first topical ophthalmic CA II inhibitor, dorzolamide, was approved under the trade name Trusopt® in the US and in combination with timolol with the trade name Cosopt® for the treatment of glaucoma or ocular hypertension. Both Cosopt® and Trusopt® are available as sterile, isotonic, buffered, slightly viscous, aqueous solution.

Brinzolamide is another carbonic anhydrase II (CA-II) inhibitor, chemically known as (R)-(+)-4-Ethylamino-2-(3-methoxypropyl)-3,4-dihydro-2H-thieno[3,2-e]-1,2-thiazine-6-sulfonamide-1,1-dioxide and is disclosed in U.S. Pat. No. 5,378,703.

Brinzolamide is soluble only to about 0.04 at physiologic pH and room temperature. Therefore a suspension dosage form was designed and the product is formulated as suspension in purified water based vehicle.

Brinzolamide ophthalmic suspension is commercially available in the US under the Trade name Azopt® and is supplied as a sterile, aqueous suspension of brinzolamide which has been formulated to be readily suspended and slow settling, following shaking. It has a pH of approximately 7.5 and an osmolality of 300 mOsm/kg. Each mL of Azopt® (brinzolamide ophthalmic suspension) 1% contains brinzolamide 10 mg as active ingredient and inactive ingredients include 0.1 mg benzalkonium chloride as a preservative, edetate disodium as a preservative and chelating agent, sodium chloride and mannitol as tonicity agents, tyloxapol as a wetting agent and carbomer 974P as a suspending agent and sodium hydroxide or hydrochloric acid are used for pH adjustment.

Major problems related to ophthalmic compositions are crystallization and agglomeration of active ingredients during preparation as well as during storage. This leads to non-uniformity of dose, difficulty of administration, irritation to eye due to large drug particles and/or any ocular adverse effect due to high drug concentration. Mainly crystallization of active ingredients useful for ophthalmic use like carbonic anhydrase inhibitor during preparation.

Sterilization by autoclaving leads to increase in solubility of the actives in the preparation and large crystals are formed during cool down phase. Aseptic ball milling of this final composition is not always practical. Aseptic addition of the all actives to a sterile vehicle is also not practical as the all actives cannot be sterilized by conventional means due to stability problem. Dry heat sterilization causes melting of the material. Sterilization by ethylene oxide introduces unacceptable degradation products and residues, and sterilization by gamma irradiation of micronized material produces degradation products unacceptable for regulatory filing.

Another reason for crystallization is change in pH due to addition of salts, acids or bases. During storage, secondary particles are formed due to partial agglomeration of suspended particles or caking. Such formation of secondary particles or caking causes problems in terms of particle size and re-dispersibility.

To overcome the above mentioned problems the following patents/patent publications disclose the different process for the preparation of sterile brinzolamide suspensions.

U.S. Pat. No. 6,071,904 discloses process for making brinzolamide ophthalmic suspension by autoclaving a milling slurry comprising brinzolamide, milling beads and surfactant followed by ball milling the milling slurry and combining aseptically this milling slurry with the autoclaved vehicle concentrate obtained by mixing polymer slurry and a solution comprising tonicity and preservative agents.

WO 2011/067791 discloses an alternate process for the preparation of brinzolamide sterile suspension comprising the steps:

(i) single stage autoclaving and sizing the final dispersion by micro fluidizer which includes combining the slurry of brinzolamide and surfactant with vehicle concentrate obtained by mixing polymer slurry and a solution comprising tonicity and preservative agents and autoclaving the said mixture followed by sizing (or)

(ii) two stage autoclaving and sizing the drug concentrate using micro fluidizer which includes autoclaving a milling slurry comprising brinzolamide, milling beads and surfactant followed by sizing of said particles in the slurry by microfluidizer and combining aseptically this slurry with the autoclaved vehicle concentrate obtained by mixing polymer slurry and a solution comprising tonicity and preservative agents.

EP 2 394 637 disclose the use of brinzolamide sterilized by gamma irradiation or ethylene oxide for manufacturing sterile ophthalmic suspensions.

WO 2012/053011 discloses process of manufacturing sterile ophthalmic suspensions comprising brinzolamide by aseptic methods and does not involve autoclaving.

The above prior art references discloses different process for the preparation of sterile suspension of brinzolamide. However, the inventors of the present invention have endeavored to develop alternate process.

### Objective of the Invention

Accordingly, the main objective of the present invention is to provide a simple and cost effective process for preparing sterile ophthalmic suspension of carbonic anhydrase inhibitor, such as brinzolamide.

### BRIEF DESCRIPTION THE DRAWINGS

FIG. 1: Flow chart showing the process for preparing ophthalmic suspension of brinzolamide, wherein the polymer slurry is combined with the solution of mannitol, tyloxopol etc., autoclaved the above dispersion and sterile brinzolamide was added. (Process-I).

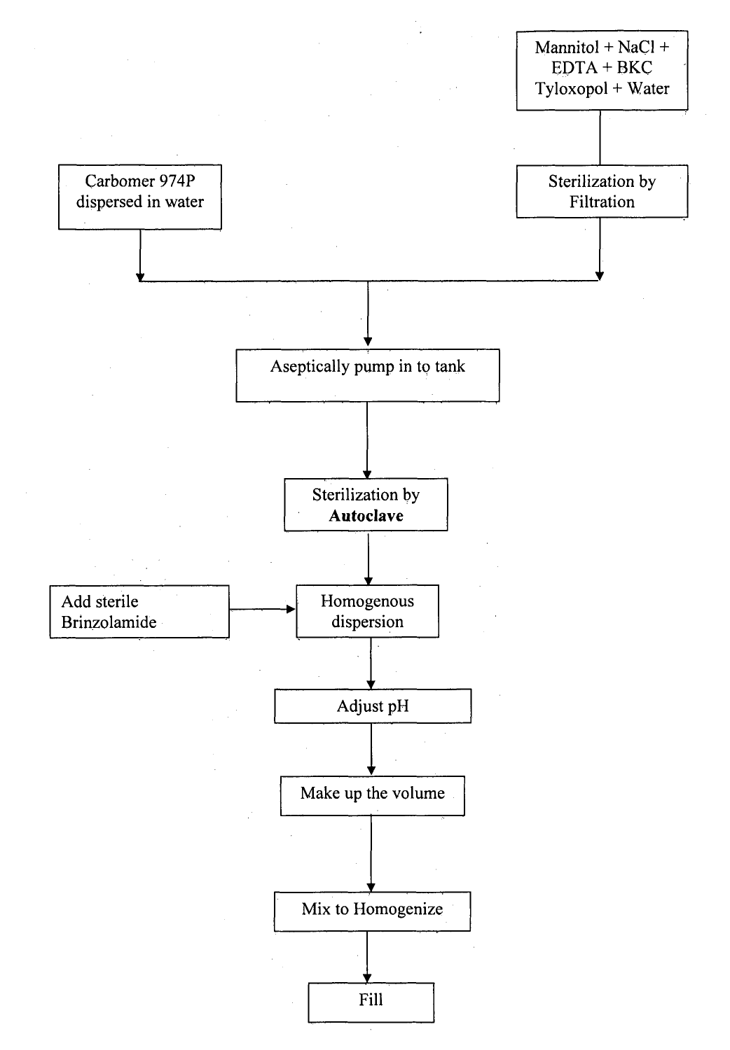

FIG. 2: Flow chart showing the process for preparing ophthalmic suspension of brinzolamide, wherein the polymer slurry is added to the slurry of brinzolamide and tyloxopal and is sterilized by autoclave and then combined with the solution of mannitol, sodium chloride etc., i.e. sterilized by autoclave. (Process-II).

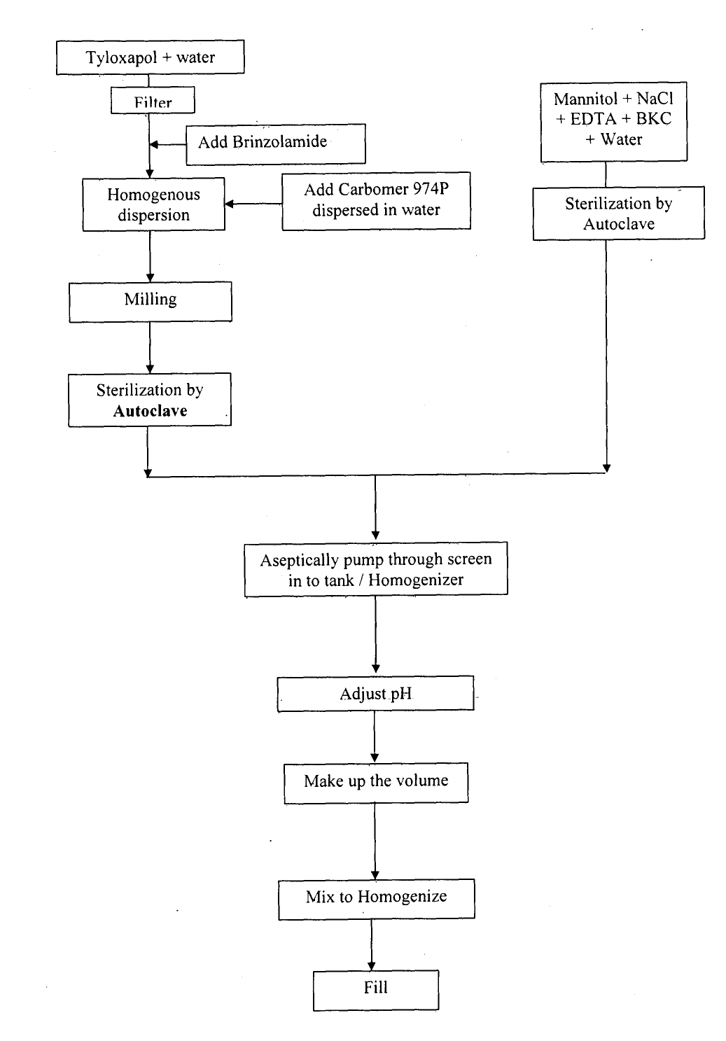

FIG. 3: Flow chart showing the process for preparing ophthalmic suspension of brinzolamide, wherein the polymer slurry is added to the slurry of brinzolamide and tyloxopal and is sterilized by autoclave and then combined with the solution of mannitol, sodium chloride etc., i.e. sterilized by aseptic filtration. (Process-III).

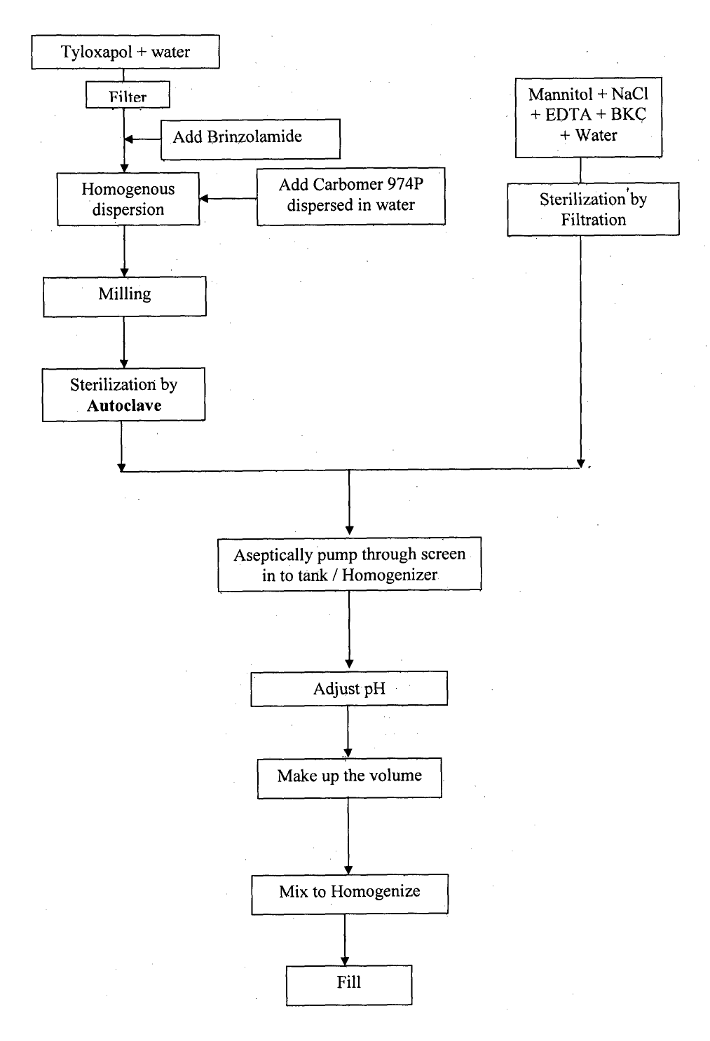

### SUMMARY OF THE INVENTION

Accordingly, the present invention provides process for preparing sterile ophthalmic suspension comprising brinzolamide as the active agent and one or more pharmaceutically acceptable excipients selected from a group comprising surfactants, polymers, preservatives, tonicity agents, pH modifiers, solvent and chelating agents.

In another embodiment, the present invention provides a process for preparing sterile ophthalmic suspension of brinzolamide comprising the steps of:

(a) preparing a slurry comprising a polymer and water,
(b) preparing a solution comprising surfactant, tonicity and preservative agents,
(c) sterilizing the solution of step (b) by aseptic filtration,
(d) aseptically combining the polymer slurry of step (a) and the solution of step
(c) to form a dispersion,
(e) sterilizing the dispersion of step (d),
(f) adding the sterile brinzolamide to autoclaved dispersion of step (e) to form the suspension and adjusting the pH,
(g) homogenizing the suspension of step (f) under aseptic conditions.

In yet another embodiment, the present invention provides a process for preparing sterile ophthalmic suspension of brinzolamide comprising the steps of:

(a) preparing a slurry comprising brinzolamide and a surfactant,
(b) preparing a slurry comprising a polymer and water,
(c) combining the slurry of step (a) and step (b) and milling the said slurry,
(d) sterilizing the homogenized slurry of step (c).
(e) preparing a solution comprising tonicity and preservative agents,
(f) sterilizing the solution of step (e),
(g) aseptically combining the slurry of step (d) and the solution of step (f) to form a suspension and adjusting the pH,
(h) homogenizing the suspension of step (g) under aseptic conditions.

### DETAILED DESCRIPTION OF THE INVENTION

In another embodiment, brinzolamide is sterilized by dry heat, gamma radiation, ethylene oxide sterilization or autoclaving.

In another embodiment, wherein the sterilization is done by using a method selected from a group comprising filtration, autoclaving, heating, irradiation, and combination thereof.

The present invention comprises a solvent wherein brinzolamide is dispersed in said solvent comprising aqueous solvent such as water.

In another embodiment the solvent system may contain one or more excipients selected from chelating agent, surfactant, preservative and pH adjusting agent.

In another embodiment, brinzolamide may be present in an amount of from about 0.001 w/v % to 10.0 w/v % and the particle size may range from 1 to 10 microns more preferably less than 5 microns.

Suitable polymers used according to the present invention are selected from a group comprising carboxyvinyl polymer (Carbopol-974P), povidone, hydroxypropylmethylcellulose, hydroxypropylcellulose, methyl cellulose, ethyl cellulose, hydroxyethylcellulose and mixtures thereof. The amount of polymer may range from about 0.01 to 2.0 w/v %.

The surfactant used according to present invention is non-toxic, non-irritant and applicable to the eye. Suitable surfactants are selected from a group comprising Tyloxapol®, Triton X-100®, polysorbates, polyoxyl 35 castor oil, polyoxyl 40 hydrogenated castor oil, polyoxyl 40 stearates, sorbitan esters, polyoxyethylene sorbitan esters, sorbitan monolaurates, sodium lauryl sulphate, polyethylene-propylene glycol copolymer (poloxamer), cremophor-40, propylene glycol, polyvinyl alcohol and mixtures thereof. The amount of surfactant may range from about 0% to 1.0 w/v %.

Suspensions that are isotonic with the lachrymal secretions cause minimal discomfort to the corneal tissue. Hence, the tonicity of the suspension has to be adjusted to maintain the formulation at an osmolality of at least about 300 mOsmol/kg. Suitable tonicity agents are selected from the group comprising mannitol, sorbitol, dextrose, dextran, glucose, glycerin, potassium chloride, sodium chloride and mixtures thereof.

Ophthalmic formulations are typically packed in multidose form. In order to inhibit the growth of microbial contaminants and suppress biodegradation preservatives are added. Suitable preservative is selected from a group comprising benzalkonium chloride, benzethonium chloride, chlorhexidine gluconate, chlorobutanol, benzyl alcohol, sodium dehydroacetate, p-hydroxybenzoate and mixtures thereof and the like. The amount of preservatives may range from about 0% to 1.0 w/v %.

Suitable pH modifiers are selected from a group comprising hydrochloric acid, acetic acid, phosphoric acid, sodium hydroxide, potassium hydroxide, ammonium hydroxide and mixtures thereof.

Suitable chelating agent is selected from a group comprising edetic acid, edetic acid salts like disodium edetate, sodium edetate, edetate calcium and trisodium edetate and the like.

In another embodiment, the present invention also provides sterile ophthalmic suspension prepared by the process as described herein comprising brinzolamide, polymer, surfactant and one or more excipients and optionally a beta blocker such as atenolol, timolol, betaxolol, arteolol and/or alpha adrenergic agonist such as brimonidine, epinephrine.

According to a preferred embodiment, the present invention provides Brinzolamide ophthalmic suspensions comprising Brinzolamide, Tyloxapol®; Carbopol® 974 P; mannitol, sodium chloride, Edetate disodium, benzalkonium chloride; sodium Hydroxide and/or Hydrochloric acid (to adjust the pH) wherein the said ophthalmic suspension is prepared by the process as described herein.

In another embodiment homogenization is done using ball mill, colloidal mill or microfluidizer for sizing of particles in the suspension.

In another embodiment, the present invention provides a process for preparing sterile ophthalmic suspension of brinzolamide comprising the steps of:

(a) preparing a slurry comprising brinzolamide and a surfactant,
(b) preparing a slurry comprising a polymer and water,
(c) combining the slurry of step (a) and step (b) and milling the said slurry,
(d) autoclaving the homogenized slurry of step (c),
(e) preparing a solution comprising tonicity and preservative agents,
(f) autoclaving the solution of step (e),
(g) aseptically combining the slurry of step (d) and the solution of step (0 to form a suspension and adjusting the pH,
(h) homogenizing the suspension of step (g) under aseptic conditions.

In yet another embodiment, the present invention provides a process for preparing sterile ophthalmic suspension of brinzolamide comprising the steps of:

(a) preparing a slurry comprising brinzolamide and a surfactant,
(b) preparing a slurry comprising a polymer and water,
(c) combining the slurry of step (a) and step (b) and milling the said slurry,
(d) autoclaving the homogenized slurry of step (c),
(e) preparing a solution comprising tonicity and preservative agents,
(f) sterilizing the solution of step (e) by aseptic filtration,
(g) aseptically combining the slurry of step (d) and the solution of step (0 to form a suspension and adjusting the pH,
(h) homogenizing the suspension of step (g) under aseptic conditions.

In a preferred embodiment, the present invention provides a process for preparing sterile ophthalmic suspension of brinzolamide comprising the steps of:

(a) preparing a slurry comprising a carbomer and water,
(b) preparing a solution comprising tyloxopol, mannitol, sodium chloride and benzalkonium chloride,
(c) sterilizing the solution of step (b) by aseptic filtration,
(d) aseptically combining the slurry of step (a) and the solution of step (c) to form a dispersion,
(e) autoclaving the dispersion of step (d),
(f) adding the sterile brinzolamide to autoclaved dispersion of step (e) to form the suspension and adjusting the pH,
(g) homogenizing the suspension of step (f) under aseptic conditions.

In another preferred embodiment, the present invention provides process for preparing sterile ophthalmic suspension of brinzolamide comprising the steps of:

(a) preparing a slurry comprising brinzolamide and tyloxopol,
(b) preparing a slurry comprising a carbomer and water,
(c) combining the slurry of step (a) and step (b) and milling the said slurry,
(d) autoclaving the homogenized slurry of step (c),
(e) preparing a solution of mannitol, sodium chloride and benzalkonium chloride in water,
(f) sterilizing the solution of step (e),
(g) aseptically combining the slurry of step (d) and the solution of step (0 to form a suspension and adjusting the pH,
(h) homogenizing the suspension of step (g) under aseptic conditions.

In another embodiment, the present invention also provides the use of brinzolamide ophthalmic suspension prepared by the process as described herein for treating glaucoma or ocular hypertension.

The following examples further exemplify the invention and are not intended to limit the scope of the invention.

### Example-1

| S.No | Ingredients |
|---|---|
| 1 | Brinzolamide |
| 2 | Benzalkonium chloride |
| 3 | Mannitol |
| 4 | Carbomer 974P |
| 5 | Sodium Chloride |
| 6 | Tyloxapol |
| 7 | Edetate Disodium |
| 8 | Hydrochloric Acid/Sodium hydroxide |
| 9 | Purified water |

The processing steps involved in manufacturing brinzolamide ophthalmic suspension given in example 1 are given below;
a) carbomer was dispersed in water to form a slurry,
b) mannitol, sodium chloride, edetate disodium, tyloxopol and benzalkonium chloride was dissolved in water,
c) the solution of step (i) was filtered,
d) the slurry of step (a) and the solution of step (b) was combined to obtain a suspension,
e) the suspension of step (d) was sterilized by autoclave,
f) sterile brinzolamide was added to the dispersion of step (e) and the pH was adjusted using Hydrochloride/Sodium hydroxide,
g) the suspension of step (f) was homogenized and then filled in dispenser.

The alternate processing steps involved in manufacturing brinzolamide ophthalmic suspension given in example 1 are given below:
a) tyloxapol was dispersed in water and then filtered,
b) brinzolamide was added to the dispersion of step (a),
c) carbomer was dispersed in water to form a slurry,
d) the dispersion of step (b) and the slurry of step (c) were combined to obtain a homigenised dispersion,
e) the dispersion of step (d) was milled and then sterilized by autoclave,
f) mannitol, sodium chloride, edetate disodium and benzalkonium chloride was dissolved in water,
g) the solution of step (f) was sterilized by autoclave,
h) combining the dispersion of step (e) and step (g) and homogenizing said dispersion and the pH was adjusted using Hydrochloride/Sodium hydroxide,
i) the suspension of step (h) was homogenized and then filled in dispenser.

The alternate processing steps involved in manufacturing brinzolamide ophthalmic suspension given in example 1 are given below:
a) tyloxapol was dispersed in water and then filtered,
b) brinzolamide was added to the dispersion of step (a),
c) carbomer was dispersed in water to form a slurry,
d) the dispersion of step (b) and the slurry of step (c) were combined to obtain a homigenised dispersion,
e) the dispersion of step (d) was milled and then sterilized by autoclave,
f) mannitol, sodium chloride, edetate disodium and benzalkonium chloride was dissolved in water,
g) the solution of step (f) was filtered,
h) combining the dispersion of step (e) and step (g) and homogenizing said dispersion and the pH was adjusted using Hydrochloride/Sodium hydroxide,
i) the suspension of step (h) was homogenized and then filled in dispenser.

### Example-2

| S.No | Ingredients |
|---|---|
| 1 | Brinzolamide |
| 2 | Benzalkonium chloride |
| 3 | Mannitol |
| 4 | Hydroxypropylmethylcellulose |
| 5 | Sodium Chloride |
| 6 | Tyloxapol |
| 7 | Edetate Disodium |
| 8 | Hydrochloric Acid/Sodium hydroxide |
| 9 | Purified water |

### Example-3

| S.No | Ingredients |
|---|---|
| 1 | Brinzolamide |
| 2 | Benzalkonium chloride |
| 3 | Mannitol |
| 4 | Carbomer 974P |
| 5 | Sodium Chloride |
| 6 | Polysorbate |
| 7 | Edetate Disodium |
| 8 | Hydrochloric Acid/Sodium hydroxide |
| 9 | Purified water |

### Example-4

| S.No | Ingredients |
|---|---|
| 1 | Brinzolamide |
| 2 | Benzalkonium chloride |
| 3 | Mannitol |
| 4 | Hydroxypropylmethylcellulose |
| 5 | Sodium Chloride |
| 6 | Polysorbate |
| 7 | Edetate Disodium |
| 8 | Hydrochloric Acid/Sodium hydroxide |
| 9 | Purified water |

The processing steps involved in manufacturing brinzolamide ophthalmic suspension as given in examples 2 to 4 is same as that of the process described in example 1.

### Example-5

| | | Formulations (%) | | | | | | | | |
|---|---|---|---|---|---|---|---|---|---|---|
| S. No | Ingredients | 5a | 5b | 5c | 5d | 5e | 5f | 5g | 5h | 5i |
| 1 | Brinzolamide | 1.00 | 1.00 | 1.00 | 1.00 | 1.00 | 1.00 | 1.00 | 1.00 | 1.00 |
| 2 | Benzalkonium | — | 0.01 | 0.01 | 0.02 | — | 0.02 | 0.005 | 0.005 | — |
| | chloride | | | | | | | | | |
| 3 | Mannitol | 2.00 | 2.00 | 2.00 | 2.50 | 2.50 | 2.50 | 2.20 | 2.20 | 2.20 |
| 4 | Carbomer 974P | 0.50 | 0.50 | 0.50 | 0.25 | 0.25 | 0.25 | 0.75 | 0.75 | 0.75 |
| 5 | Sodium Chloride | 0.80 | 0.80 | 0.80 | 0.80 | 0.80 | 0.80 | 0.80 | 0.80 | 0.80 |
| 6 | Tyloxapol | 0.10 | — | 0.10 | 0.05 | 0.05 | — | — | 0.20 | 0.20 |
| 7 | Edetate disodium | 0.01 | 0.01 | — | — | 0.02 | 0.02 | 0.025 | | 0.025 |
| 8 | Hydrochloric | q.s | q.s | q.s | q.s | q.s | q.s | q.s | q.s | q.s |
| | Acid/Sodium | | | | | | | | | |
| | hydroxide | | | | | | | | | |
| 9 | Purified water | q.s | q.s | q.s | q.s | q.s | q.s | q.s | q.s | q.s |

The processing steps involved in manufacturing brinzolamide ophthalmic suspension are as set forth.
a) carbomer was dispersed in water to form a slurry,
b) mannitol, sodium chloride, optionally edetate disodium and/or tyloxopol and/or benzalkonium chloride was dissolved in water,
c) the solution of step (i) was filtered,
d) the slurry of step (a) and the solution of step (b) was combined to obtain a suspension,
e) the suspension of step (d) was sterilized by autoclave,
f) sterile brinzolamide was added to the dispersion of step (e) and the pH was adjusted using Hydrochloride/Sodium hydroxide,
g) the suspension of step (f) was homogenized and then filled in dispenser.

The alternate processing steps involved in manufacturing brinzolamide ophthalmic suspension are given below:
a) tyloxapol was dispersed in water and then filtered,
b) brinzolamide was added to the dispersion of step (a),
c) carbomer was dispersed in water to form a slurry,
d) the dispersion of step (b) and the slurry of step (c) were combined to obtain a homigenised dispersion,
e) the dispersion of step (d) was milled and then sterilized by autoclave, mannitol, sodium chloride, optionally edetate disodium and/or benzalkonium chloride was dissolved in water,
g) the solution of step (f) was sterilized by autoclave,
h) combining the dispersion of step (e) and step (g) and homogenizing said dispersion and the pH was adjusted using Hydrochloride/Sodium hydroxide,
i) the suspension of step (h) was homogenized and then filled in dispenser.

The alternate processing steps involved in manufacturing brinzolamide ophthalmic suspension are given below:
a) tyloxapol was dispersed in water and then filtered,
b) brinzolamide was added to the dispersion of step (a),
c) carbomer was dispersed in water to form a slurry,
d) the dispersion of step (b) and the slurry of step (c) were combined to obtain a homigenised dispersion,
e) the dispersion of step (d) was milled and then sterilized by autoclave,
f) mannitol, sodium chloride optionally edetate disodium and/or benzalkonium chloride was dissolved in water,
g) the solution of step (f) was filtered,
h) combining the dispersion of step (e) and step (g) and homogenizing said dispersion and the pH was adjusted using Hydrochloride/Sodium hydroxide,
i) the suspension of step (h) was homogenized and then filled in dispenser.

The alternate processing steps involved in manufacturing brinzolamide ophthalmic suspension are given below:
a) brinzolamide was dispersed in water,
b) carbomer was dispersed in water to form a slurry,
c) the dispersion of step (a) and the slurry of step (b) were combined to obtain a homigenised dispersion,
d) the dispersion of step (c) was milled and then sterilized by autoclave,
e) mannitol, sodium chloride, optionally edetate disodium and/or benzalkonium chloride was dissolved in water,
f) the solution of step (e) was sterilized by autoclave,
g) combining the dispersion of step (d) and step (f) and homogenizing said dispersion and the pH was adjusted using Hydrochloride/Sodium hydroxide,
h) the suspension of step (g) was homogenized and then filled in dispenser.

The alternate processing steps involved in manufacturing brinzolamide ophthalmic suspension are given below:
a) brinzolamide was dispersed in water,
b) carbomer was dispersed in water to form a slurry,
c) the dispersion of step (a) and the slurry of step (b) were combined to obtain a homigenised dispersion,
d) the dispersion of step (c) was milled and then sterilized by autoclave,
e) mannitol, sodium chloride, optionally edetate disodium and/or benzalkonium chloride was dissolved in water,
f) the solution of step (e) was filtered,
g) combining the dispersion of step (d) and step (f) and homogenizing said dispersion and the pH was adjusted using Hydrochloride/Sodium hydroxide,
h) the suspension of step (g) was homogenized and then filled in dispenser.

### Example-6

| S.No | Ingredients |
|---|---|
| 1 | Brinzolamide |
| 2 | Timolol maleate |
| 3 | Benzalkonium chloride |
| 4 | Mannitol |
| 5 | Carbomer 974P |
| 6 | Sodium Chloride |
| 7 | Tyloxapol |
| 8 | Edetate Disodium |
| 9 | Hydrochloric Acid/Sodium hydroxide |
| 10 | Purified water |

The processing steps involved in manufacturing brinzolamide ophthalmic suspension given in example 1 are given below:
h) carbomer was dispersed in water to form a slurry.
i) mannitol, sodium chloride, edetate disodium, tyloxopol and benzalkonium chloride, timolol maleate was dissolved in water,
j) the solution of step (i) was filtered,
k) the slurry of step (a) and the solution of step (b) was combined to obtain a suspension,
l) the suspension of step (d) was sterilized by autoclave,
m) sterile brinzolamide was added to the dispersion of step (e) and the pH was adjusted using Hydrochloride/Sodium hydroxide,
n) the suspension of step (f) was homogenized and then filled in dispenser.

The alternate processing steps involved in manufacturing brinzolamide ophthalmic suspension given in example 1 are given below:
j) tyloxapol was dispersed in water and then filtered,
k) brinzolamide was added to the dispersion of step (a),
l) carbomer was dispersed in water to form a slurry,
m) the dispersion of step (b) and the slurry of step (c) were combined to obtain a homigenised dispersion,
n) the dispersion of step (d) was milled and then sterilized by autoclave,
o) mannitol, sodium chloride, timolol maleate, edetate disodium and benzalkonium chloride was dissolved in water,
p) the solution of step (f) was sterilized by autoclave,
q) combining the dispersion of step (e) and step (g) and homogenizing said dispersion and the pH was adjusted using Hydrochloride/Sodium hydroxide,
r) the suspension of step (h) was homogenized and then filled in dispenser.

The alternate processing steps involved in manufacturing brinzolamide ophthalmic suspension given in example 1 are given below:
j) tyloxapol was dispersed in water and then filtered,
k) brinzolamide was added to the dispersion of step (a),
l) carbomer was dispersed in water to form a slurry,
m) the dispersion of step (b) and the slurry of step (c) were combined to obtain a homigenised dispersion,
n) the dispersion of step (d) was milled and then sterilized by autoclave,
o) mannitol, sodium chloride, timolol maleate, edetate disodium and benzalkonium chloride was dissolved in water,
p) the solution of step (f) was filtered,
q) combining the dispersion of step (e) and step (g) and homogenizing said dispersion and the pH was adjusted using Hydrochloride/Sodium hydroxide,
r) the suspension of step (h) was homogenized and then filled in dispenser.

1. Process for preparing sterile ophthalmic suspension comprising brinzolamide as the active agent and one or more pharmaceutically acceptable excipients selected from a group comprising surfactants, polymers, preservatives, tonicity agents, pH modifiers, solvent and chelating agents.

2. A process for preparing sterile ophthalmic suspension of brinzolamide according to claim 1, comprising the steps of:
(a) preparing a slurry comprising a polymer and solvent,
(b) preparing a solution comprising one or more of surfactant, tonicity agent and preservative,
(c) sterilizing the solution of step (b),
(d) aseptically combining the polymer slurry of step (a) and the solution of step (c) to form a dispersion,
(e) sterilizing the dispersion of step (d),
(f) adding the sterile brinzolamide to dispersion of step (e) to form the suspension, and optionally adjusting the pH,
(g) homogenizing the suspension of step (f) under aseptic conditions.

3. A process for preparing sterile ophthalmic suspension of brinzolamide according to claim 1, comprising the steps of:
(a) preparing a slurry comprising brinzolamide and a surfactant,
(b) preparing a slurry comprising a polymer and solvent,
(c) combining the slurry of step (a) and step (b) and milling the said slurry,
(d) sterilizing the homogenized slurry of step (c),
(e) preparing a solution comprising tonicity agent and preservative,
(f) sterilizing the solution of step (e),
(g) aseptically combining the slurry of step (d) and the solution of step (f) to form a suspension, and optionally adjusting the pH,
(h) homogenizing the suspension of step (g) under aseptic conditions.

4. The process according to claim 2, wherein the sterilization in steps (c) and (e) is done by using a method selected from a group comprising filtration, autoclaving, heating, irradiation, and combination thereof.

5. The process according to claim 3, wherein the sterilization in steps (d) and (f) is done by using a method selected from a group comprising filtration, autoclaving, heating, irradiation, and combination thereof.

6. The process according to any of the claims 2-3, wherein the pH is adjusted using a pH adjusting agent.

7. The process according to claim 6, wherein the pH adjusting agent is selected from a group comprising hydrochloric acid, acetic acid, phosphoric acid, sodium hydroxide, potassium hydroxide, ammonium hydroxide and mixtures thereof.

8. The process according to any of the preceding claims 1-7, wherein the polymer is selected from a group comprising carboxyvinyl polymer, povidone, hydroxypropylmethylcellulose, hydroxypropylcellulose, methyl cellulose, ethyl cellulose, hydroxyethylcellulose, and mixtures thereof.

9. The process according to any of the preceding claims 1-8, wherein the surfactant is selected from a group comprising tyloxapol, polysorbates, polyoxyl 35 castor oil, polyoxyl 40 hydrogenated castor oil, polyoxyl 40 stearates, sorbitan esters, polyoxyethylene sorbitan esters, sorbitan monolaurates, sodium lauryl sulphate, polyethylene-propylene glycol copolymer, propylene glycol, polyvinyl alcohol, and mixtures thereof.

10. The process according to any of the preceding claims 1-9, wherein the preservative is selected from a group comprising benzalkonium chloride, benzethonium chloride, chlorhexidine gluconate, chlorobutanol, benzyl alcohol, sodium dehydroacetate, p-hydroxybenzoate, and mixtures thereof.

11. The process according to any of the preceding claims 1-10, wherein the tonicity agent is selected from a group comprising mannitol, sorbitol, dextrose, glucose, glycerin, potassium chloride, sodium chloride.

12. The process according to any of the preceding claims 1-11, wherein the solvent is an aqueous solvent.

13. The process according to any of the preceding claims 1-11, wherein the solvent is water.

14. A process according to any of the preceding claims 1-13, wherein the quantity of surfactant ranges from 0% to about 10%, polymer ranges from about 0.5% to about 1.0%, preservative ranges from 0% to about 1%, tonicity agents range from 0% to about 10%, pH modifier ranges from 0% to about 10%, chelating agent ranges from 0% to about 5%, and solvent ranges from about 10% to about 99% w/v of the suspension.

15. An ophthalmic suspension comprising brinzolamide, surfactant, polymer, tonicity agent, preservative, water, and optionally pH adjusting agent, wherein the said ophthalmic suspension is prepared according to the process as claimed in claim 2.

16. An ophthalmic suspension according to claim 15, comprising brinzolamide, tyloxapol, carbopol, mannitol, sodium chloride, edetate disodium, benzalkonium chloride, sodium hydroxide, and optionally hydrochloric acid.

17. An ophthalmic suspension comprising brinzolamide, surfactant, polymer, tonicity agent, preservative, water, and optionally pH adjusting agent, wherein the said ophthalmic suspension is prepared according to the process as claimed in claim 3.

18. An ophthalmic suspension according to claim 17, comprising brinzolamide, tyloxapol, carbopol, mannitol, sodium chloride, edetate disodium, benzalkonium chloride, sodium hydroxide, and optionally hydrochloric acid.

19. Use of brinzolamide ophthalmic suspension prepared according to any of the claims 1-3, for treating glaucoma or ocular hypertension.
